(19) United States
(12) Patent Application Publication
(10) Pub. No.: US 2018/0291324 A1
(43) Pub. Date: Oct. 11, 2018

(54) BIOMIMETIC AMNIOTIC MEMBRANE NICHE FOR STEM CELLS

(71) Applicant: SCIENCE & TECHNOLOGY DEVELOPMENT FUND, Cairo (EG)

(72) Inventors: Ahmed Al-Sayed Noha MOUSA, Assiut (EG); Mohamed Ahmed Sabreen KHALIL, Assiut (EG); El-Sayed Mohamed Nagwa EL-BADRI, Cairo (EG)

(21) Appl. No.: 15/739,172

(22) PCT Filed: Jun. 25, 2015
(86) PCT No.: PCT/EG15/00030

§ 371 Date: Dec. 22, 2017

(51) Int. Cl. C12M 1/12; C12M 1/00; C12M 1/24; C12M 1/36; A61L 27/36; A61L 27/38; C12M 3/06; A61L 27/58; C12N 5/0775

(52) U.S. Cl. CPC C12M 25/02; C12M 23/26; C12M 23/08; C12M 29/10; C12M 29/04; C12M 41/48; C12N 2503/00; A61L 27/3834; C12M 23/16; C12M 23/30; A61L 27/58; C12N 5/0665; C12N 2533/92; A61L 27/3604

(57) ABSTRACT

In this invention we propose a method to compose a stem cell culture niche platform, which is based on the use of the human amniotic membrane. Fluid dynamic, mechanical and topographic factors are additionally included in this niche to provide various factors essential for achieving an enhanced biomimetic microenvironment of the cultured stem cells. The amniotic membrane is mounted into various types of culture platforms to suit a wide range of research applications. The rich composition of the membrane with anti-inflammatory, anti microbial, matrix and adhesion molecules in addition to various growth factors suits its application as a complex biomimetic material. The platform includes micro channels to allow continuous exchange of media and creates a dynamic flow of the fluid surrounding the cells in an attempt to simulate the in vivo conditions in which the stem cell typically reaches its ideal proliferation, expansion or differentiation. The method disclosed herein supports a wide range of applications in stem cell research such as the investigation of the optimal conditions for stem cell culture and the effect of various medications and external factors. It can be also applied in investigating the effect of the amniotic membrane and the mechanical factors on the behavior of stem cells and cancer stem cells. Another model of the niche is proposed as an in vivo moldable and implantable carrier for delivering stem cell based therapies in a wide range of diseases especially those associated with aging or decline of specialized cell function such as diabetes, cardiovascular, neurological, hormonal, renal and liver disorders, cancer, and diseases associated with inflammation and disordered immunity. Furthermore, the lack of HLA molecules renders the membrane naïve to minimize rejection, which could be valuable for transplantation purposes.

### TECHNICAL FIELD

The invention relates to the field of cell biotechnology, stem cell culture, mammalian cell culture, biomimetic systems, biomaterials, and stem cell therapy and cell transplantation.

Particularly, the present invention relates to a method of composing platforms that act as biomimetic niche for stem cell culture and, stem cell research and clinical applications.

### BACKGROUND ART

The amniotic membrane has been used for decades as a surgical graft. Several inventions such as the patent by Scheffer C G (WO1998037903 A1 and U.S. Pat. No. 6,326,019 B1) focused on the preparation and the use of the amniotic membrane for surgical applications. It has been used widely as an ophthalmic surgical graft and as a substrate for ocular surface reconstruction allowing ex vivo expansion of limbal stem cells prior to its transplantation in patients (WO 2003077794 A3). It was also proposed as a biopolymer that can be used as a bandage or as contact lens to cover the ocular surface to promote healing and provide antimicrobial protection (WO 2003077794 A3).

The amniotic membrane was lyophilized or dry-frozen into a powder followed by its use in various forms including eye drops, ointments and gels for medical applications especially eye disorders, and for its regenerative and anti-aging properties (US 20040057938 A1). Lyophilization of the membrane to allow its long-term preservation under various temperatures and conditions was also recently proposed (US 20140186461 A1). Similarly, the amniotic membrane was proposed as a scaffold for culturing amniotic stem cells and differentiating them to keratinocytes to compose a skin substitute that can be used in burn repairs and other skin defects (U.S. Pat. No. 8,287,854 B2). On another front, a factor that is extracted from the amniotic membrane was proposed for the enhancement of culture of human embryonic stem cells (EP 1767619 A4). In a novel strategy, the amniotic membrane was used as is, without decellularization, to differentiate its stem cell content in situ into osteogenic cells for the purpose of bone regeneration (EP 2576767 A1). Furthermore, the compounding of the amniotic membrane with different consistency modifying polymers was proposed to form an implantable device that can be used inside the body (WO 2006002128 A1).

Stem cells are capable of unlimited cell division and of differentiating into other specialized cell and tissue types, each of which can serve unique function. They play vital roles in human development. In adult life, they maintain homeostatic regeneration of tissues and repair injured body organs. Stem cells are currently obtained from various sources including human embryonic stem cells, adult tissue, umbilical cord blood or matrix, placenta, induced pluripotent stem cells and cancer stem cells.

Based on the central potential role in human health, stem cell research is unprecedentedly expanding worldwide. Stem cell-based therapies are currently investigated in the treatment of diabetes, cardiac diseases, brain and neurological disorders, liver disease, cancer, genetic disorders and many others. Furthermore, stem cells are increasingly included in the production of bio scaffolds of engineered tissue constructs for organ replacement applications. Accordingly, a broad range of significant diseases affecting humans are dependent on the proper identification of stem cells and the consequent ability to either use or target these cells.

The stem cell niche encompasses all biological, physical, micro architectural and biomechanical factors that contribute to the natural microenvironment of the cell. Such factors define how a stem cell behaves under various circumstances and when and how it could differentiate in response to any particular internal or external stimulus (Scadden D T, 2014). These factors could also affect expansion of stem cells, programmed cell death or the transformation into a malignant progeny leading to a population of cancer stem cells or tumor initiating cells. Consequently, there has been an increasing attention towards the stem cell niche and how the in vitro niche could be composed in a way that mimics the actual in vivo microenvironment of the stem cell. The topographic, physical and mechanical factors are among several important contributors believed to affect the fate of stem cells (Guilak, Cohen et al. 2009).

Since the majority of stem cell studies have been conducted in laboratory conditions using artificial platforms and static culture media, the outcome and conclusions of many of these studies fail to prove clinically relevant when stem cells are transplanted in vivo for therapeutic applications. Expansion of stem cells remains a challenge to generate a clinically useful number of undifferentiated cells suitable for further differentiation and cell therapy. Likewise, homing and engraftment of stem cells by the target organ in vivo pose other hurdles that remain to be overcome (Eggenhofer, Benseler et al. 2012).

Accordingly, these challenges necessitated the production of new environments that simulate the natural stem cell niche and its basic elements (Lutolf et al. 2009).

It is now agreed that the current in vitro methods of stem cell culture, expansion and differentiation are suboptimal and notably far from conditions needed for accurate reproduction or simulation of the in vivo stem cell niche needed for effective stem cell-based therapies. The extracellular matrix (ECM) surrounding cells in the body provides complex groups of proteins, growth factors, adhesion molecules, transport molecules necessary for important cellular functions and cell-to-cell interaction (Summa P G D et al, 2013). It also provides the mechanical skeleton and three-dimensional surface topography that allows cells to interact and align together to form functional tissue units that are characteristics of each organ. Conventional culture plates used by stem cell researchers provide no extracellular matrix but only a flat two-dimensional artificial plastic based surface. More recently available culture plates are coated mostly with a single extracellular matrix protein such as plates coated with collagen, fibronectin or laminin. Other plates are coated with nano fibers to provide a structural network for cells to grow and align on. These plates are of significant cost compared to the non-coated regular plates and do not yet provide all other factors of the real in vivo stem cell niche.

The human amniotic membrane (hAM) is composed of a complex of numerous ECM molecules and cells that could provide support to the growing stem cells. The selection of the human amniotic membrane as a component of our proposed stem cell culture niche is based on a robust clinical and research evidence of its unique properties as a biomaterial that have been used as a surgical grafting for several years.

Multiple components of the hAM were demonstrated previously including the expression of diverse growth factors such as KGF, b-FGF, HGF and TGF-β. Its intact collagen fibers are believed to provide better cellular migration and alignment (Koizumi, 2000).

Antimicrobial and anti-inflammatory properties have been also attributed to several components of the human amniotic membrane including matrix metalloproteinases (MMPs) inhibitors, elafin and leukocyte proteinase inhibitor (SLPPI). Their ability to suppress pro-inflammatory cytokines such as IL-1α and IL-1β was previously demonstrated. Importantly, hAM is significantly less immunogenic and should be more biocompatible than compounds extracted from adult human and animal tissues or artificial material commonly used in traditional culture systems. (Grueterich, Espana et al. 2003) (Niknejad, Peirovi et al. 2008).

### DISCLOSURE OF THE INVENTION

The present invention provides a method for developing a low cost biomimetic niche for cell culture studies and applications using a human amniotic membrane-based platform combined with biomechanical and topographic properties. The cells of interest are preferred to be stem cells (for example embryonic stem cells, adult stem cells, placental stem cells, cord blood stem cells, cord matrix stem cells, cancer stem cells and induced pluripotent stem cells). The method can be also applied for studies of other cells and microorganisms. It can be also used for explant tissue culture where tiny fragments of tissues are embedded in the proposed culture system and explanted cells proliferate and expand out of the tissue fragments.

In this invention, we use the hAM as an integral part of a more comprehensive niche. The hAM will provide essential biological and physical factors; in addition to a low cost xeno-free easily prepared three-dimensional topography required for a biomimetic niche. The invention additionally includes fluid dynamic factors that together provide a novel module that could mimic in vivo system conditions.

Preparation of the Human Amniotic Membrane (hAM)

Human amniotic membrane specimens were obtained from healthy women after delivery and clamp-cutting of the umbilical cord. The institutional research board approved using products of delivery for the purpose of research. Participants approved using their samples in the study through an informed consent. Women who screened negative for blood borne infection were included. The amnion was washed with sterile saline solution then separated manually or by blunt dissection from the chorion layer of the amniotic membrane. It was important to mark the epithelial surface of the membrane from the connective tissue throughout this work. It was then washed several times by phosphate buffer saline (PBS) containing 0.1% antibiotic antimycotic to remove excess blood. The membrane specimen was then divided into small sections of 10 cm diameter each. The membrane was stretched on a homemade cell crown to allow homogenous distribution of the treating decellularization reagents.

Decellularization of the Human Amniotic Membrane (hAM):

In order to remove the single epithelial cell layer of the amnion, an initial investigation of previously reported methods was done. The use of trypsin EDTA with mechanical scraping was compared to the use of sodium hydroxide as reported by (Saghizadeh, 2013). For trypsin treatment, trypsin (0.25%) was used to treat the membrane for 30 minutes. For sodium hydroxide treatment, 2 grams were dissolved in 50 mL of distilled water (concentration 40 mg/mL) and was used only for 30 seconds to 1 minute on the epithelial side of the membrane, with the application of light cotton tip rubbing. The membrane was then washed for 5 to 10 minutes using PBS. Methylene blue and tryban blue were used to stain the membrane independently to examine the efficiency of cell removal by both methods and to evaluate viability of any remaining cells. The membrane was examined by bright field microscopy. Complete decellularization was achieved using the sodium hydroxide based protocol in significantly less time compared to using trypsin, which led to partial decellularization.

Surface Electron Microscope Examination (SEM):

In order to examine the ultra surface topography of the decellularized amniotic membrane and compare it with the non-treated amniotic membrane, SEM examination was employed. After treatment of the amniotic membrane by NaOH, the fresh membrane was preserved in glutaraldehyde as a fixative to provide optimal preservation of the membrane ultra structural details. The specimens were molded on a specimen stub and sputter coated with gold required for SEM examination. Images were obtained at different magnifications (×500, ×1000, ×2000, ×5000).

Prototype

Various examples of platforms were developed to optimize the conditions needed for a comprehensive prototype and to increase the scope of applicability of the disclosed invention.

### EXAMPLE 1

hAM was decellularized as described above and used to line regular commonly used plastic culture containers such as the petri dish and the 6 well plate.

The lined plates were used to test basic culture effectiveness of different stem cell types including umbilical cord blood stem cells and amniotic fluid stem cells. In this example, static culture media is used and fluid dynamics were not included to match the routine current applications without the need of extensive upgrade.

### EXAMPLE 2

A novel prototype is developed to provide all proposed niche components in one setting. The platform is fabricated using polydimethylsiloxane (PDMS) elastomer.

The PDMS allows the molding and flexibility of unlimited number of designs and shapes of the containers. As an example, a spherical container was fabricated with an average thickness of 3-5 mm. In brief, a urinary Foley's catheter balloon was inflated and inserted in a spherical soft plastic ball opened at its central top. Liquid PDMS was poured evenly all around the inflated Foley's balloon. An internal PDMS knob was made. Inlet and outlet channels were made to deliver and drain culture medium continuously by external attachment to a syringe pump. hAM was then decellularized as outlined above and suctioned to adhere firmly to the internal surface of the platform.

Another disc-shaped PDMS piece is made to hang free in the fluid medium of the prototype. The disc is patterned to a three dimensional surface that is further covered by the decellularized amniotic membrane.

### EXAMPLE 3

A basic microchannel microfluidic chip was used. hAM was decellularized as outlined above and small segments of around 5×10 mm were cut. Tiny plugs of around 2 mm sizes were cut of a firm PDMS and the amniotic membrane was applied on its convex surface. The hAM-covered plug was inserted into the upper section of a micro chamber of around 3 mm diameter that is connected to inlet and outlet microchannels (of around 200 μm). Continuous flow of fluids is achieved by either hydrostatic pressure or by automated syringe pump based flow. The walls of this microchamber and the plug can be all patterned for more complex three-dimensional surface as needed.

Isolation of Umbilical Cord Blood Derived Mononuclear Cells (MNCs):

Isolation of the mononuclear cells was performed as described previously with modifications (Bieback et al, 2004). In brief, the umbilical cord blood (volumes ranged from 20 mL to 50 mL) was collected from the umbilical cord into a heparin containing sterile tubes. Samples were diluted in 1:1 v/v using PBS. The diluted blood was centrifuged at 400 g for 30 minutes. The buffy coat of the mononuclear cells MNCs) was collected and washed twice by PBS and centrifuged at 250 g for 10 minutes to eliminate platelets. Finally, cells were suspended in 5 mL of DMEM containing 10% FBS and counted. The concentration of cells per ml was around 5.6×10⁶ cells/mL.

Culture of the MNCs

Around 1×10⁶ cells were cultured on each of the hAM-coated 6-well plates and the volume of the culture medium was 2 mL in each well. Additionally, MNCs were cultured in regular wells without hAM as a control group. The plate was incubated at 37° C. and 5% CO₂. Serial observations of cells after culture on the membrane were obtained.

Culture of Amniotic Fluid Cells

Amniotic fluid was collected by sterile syringe for the purpose of amnio reduction from pregnant women with polyhydraminos. The excess fluid planned to be discarded as a bio waste was used to obtain amniotic fluid stem cells. Amniotic fluid of various volumes (range is about 15-40 mL) was collected from different patients. Amniotic fluid was either immediately processed or cryopreserved in −80° C., or liquid nitrogen till further use. The fluid was thawed and centrifuged until a cell pellet was obtained. Cells were cultured to investigate the functional performance of different developed prototypes.

### REFERENCES

Bieback, Karen, et al. “Critical parameters for the isolation of mesenchymal stem cells from umbilical cord blood.” Stem cells 22.4 (2004): 625-634.
Eggenhofer, E., V. Benseler, et al. (2012). “Mesenchymal stem cells are short-lived and do not migrate beyond the lungs after intravenous infusion.” Front Immunol 3: 297.
Grueterich, M., E. M. Espana, et al. (2003). “Ex vivo expansion of limbal epithelial stem cells: amniotic membrane serving as a stem cell niche.” Survey of ophthalmology 48(6): 631-646.
Guilak, F., D. M. Cohen, et al. (2009). “Control of stem cell fate by physical interactions with the extracellular matrix.” Cell stem cell 5(1): 17-26.
Koizumi, Noriko, et al. “Growth factor mRNA and protein in preserved human amniotic membrane.” Current eye research 20.3 (2000): 173-177.
Lutolf, Matthias P., Penney M. Gilbert, and Helen M. Blau, “Designing materials to direct stem-cell fate.” Nature 462.7272 (2009): 433-441.
Niknejad, H., H. Peirovi, et al. (2008). “Properties of the amniotic membrane for potential use in tissue engineering.” Eur Cells Mater 15: 88-99.
Saghizadeh, Mehrnoosh, et al. “A simple alkaline method for decellularizing human amniotic membrane for cell culture.” PloS one 8.11 (2013): e79632.
Scadden, David T. “Nice neighborhood: emerging concepts of the stern cell niche.” Cell 157.1 (2014): 41-50.
Summa, P G D., et al. “Extracellular matrix molecules enhance the neurotrophic effect of Schwann cell-like differentiated adipose-derived stem cells and increase cell survival under stress conditions.” Tissue Engineering Part A 19.3-4 (2012): 368-379.

### BRIEF DESCRIPTION OF THE DRAWINGS

Preferred embodiments of the invention will now be described, by way of example only, with reference to the drawings, in which:

FIG. 1: It shows a drawing of one example of a design of the platform 1, lined by the human amniotic membrane 2, and one internal knob 3 representing said a mobile plate covered with amniotic membrane and has a three dimensional surface topography. Fluid inflow and outflow are shown 7, external connecting tubes for inflow and outflow channels are shown 4 and 5. An external source for inducing mobility 6 could be included to support the internal flow dynamics or to replace it based on the application

FIG. 2: Shows a drawing of another example of the design of the platform wherein, 12 denotes the provision of repeated units of the same platform as required by the application.

FIG. 3: Shows a drawing of cut section of a spherical design of the platform that contains an internal spherical porous knob also covered by the membrane. The fluid delivering channel 9 allows the delivery of culture media and other important constituents and is connected through the internal side of the knob. Filters are added to the fluid channels to prevent the exit of cells during the flow.

FIG. 4: Shows a drawing of an implantable system for therapeutic application and stem cell transplantation. It is composed of an external amniotic based skeleton 13 surrounding and patterned on harder posts or frame 14 to mold it for the desired shape. It can connect to the in vivo microenvironment through micro channels 16, equipped with internal built in micro filters of various sizes 15 based on the application of interest basically to either allow the exit or exchange of cells and molecules or to allow the exit of molecules and factors secreted by those cells without the passage of cells.

FIG. 5: This figure shows various prototypes that are in current development to investigate the effectiveness of the platform. P1 is for a platform that employs a regular petri dish. P2 for 6-well plate lined with amniotic membrane, P3 shows successive steps of developing a distinct prototype that enables easier incorporation of surface and dynamic factors (a-d). P4 is for a prototype that is dependent on a microchip reservoir connected with micro channels (inlet and outlet microchannels fabricated inside the chip).

FIG. 6: This figure shows a surface electron microscope image of the human amniotic membrane after decellularization. The natural three-dimensional topography is well demonstrated at ×5000 magnification S1. An image J software used to further highlight this topography using an inverse surface mode S2 and a 3D surface plot function of the same image S3.

FIG. 7: This figure shows preliminary data of human umbilical cord blood mononuclear cells that was isolated and cultured on the amniotic membrane lined plates. The proliferation of cells on successive days of the culture is shown as D0, D3, 4 and 5.

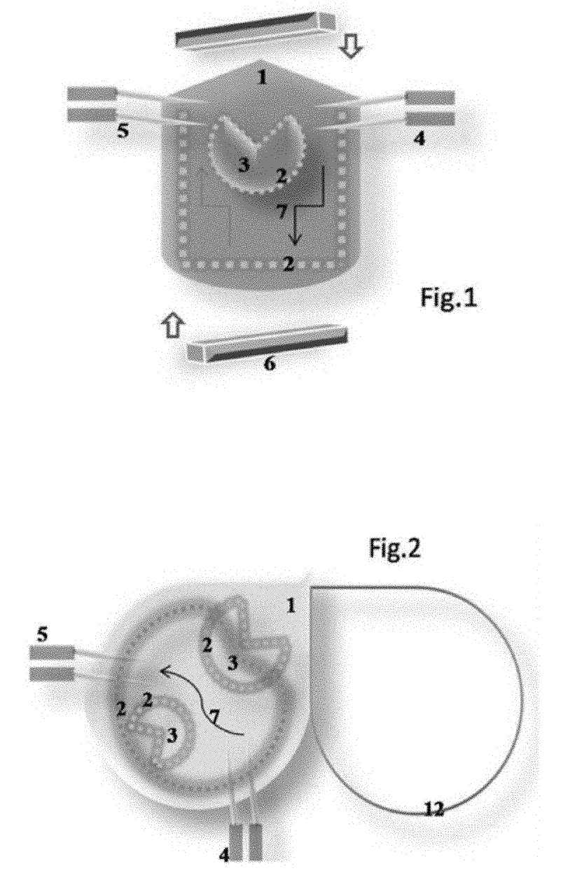

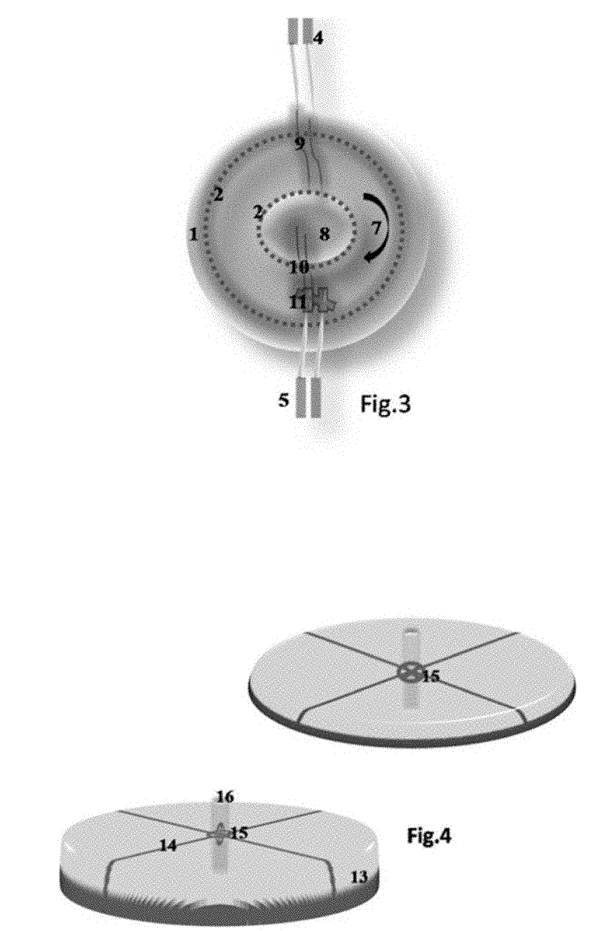

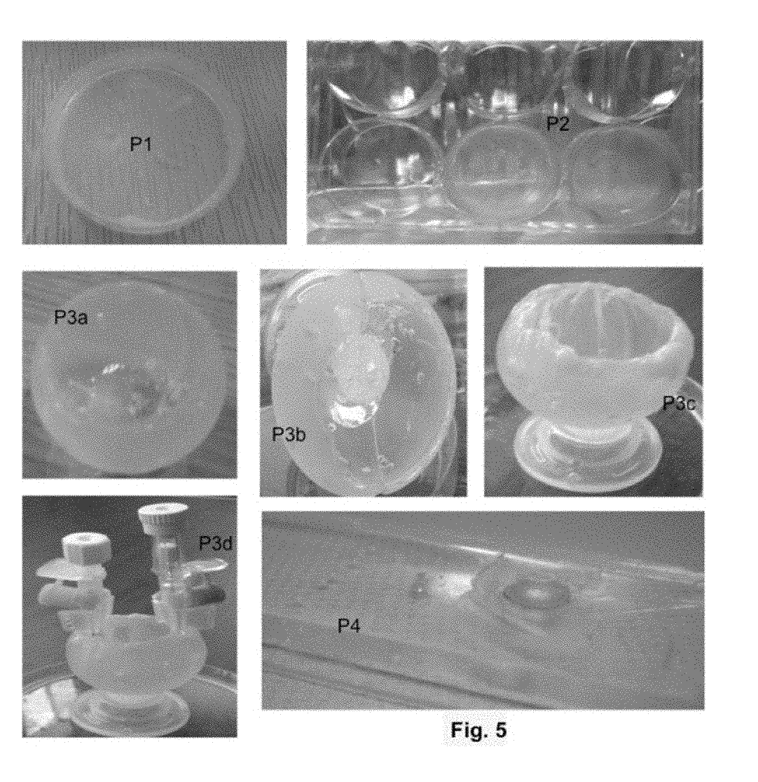

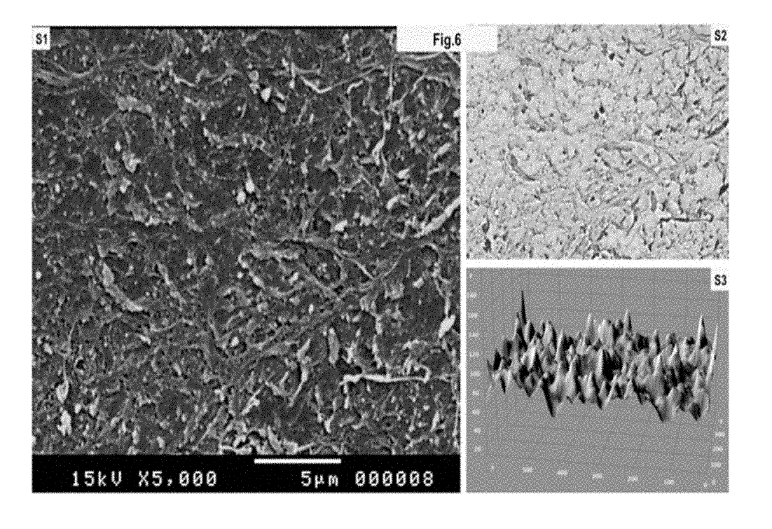

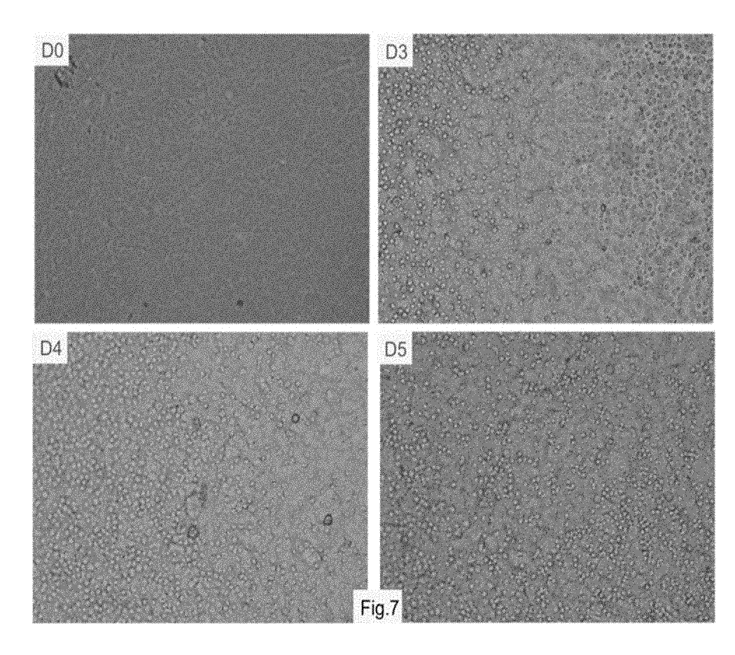

The examples and embodiments described herein are for illustrative purposes only. Modifications or changes in light thereof will be suggested to persons skilled in the art and are to be included within the spirit and purview of this application. The figures are not to scale and some features may be exaggerated or minimized to show details of particular elements while related elements may have been eliminated to prevent obscuring novel aspects. Accordingly, specific structural and functional details disclosed herein are not to be interpreted as restrictive but as a basis for the claims and as a representative basis for teaching one skilled in the art to variously employ the present invention. As used herein, the terms “about”, and “approximately” when used in conjunction with ranges of concentrations, temperatures or other physical or chemical properties or characteristics is meant to cover slight variations that may exist in the upper and lower limits of the ranges of properties/characteristics. Also, as used herein, the terms “comprises”, “comprising”, “includes” and “including” are to be construed as being inclusive and open ended, and not exclusive. Specifically, when used in this specification including claims, the terms “comprises”, “comprising”, “includes” and “including” and variations thereof mean the specified features, steps or components are included. These terms are not to be interpreted to exclude the presence of other features, steps or components. The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

1. A method of creating one or more biomimetic cell culture platform to mimic a native cell niche including the use of biological membranes and tissues embedded in three dimensional surface with a fluid dynamic mechanical factor.

2. As mentioned in claim #1, the platform could be of various shapes and sizes including flasks, plates, dishes, wells, dishes, channels, vessels, capsules, discs or any custom made containers

3. As mentioned in claim #1, the biomimetic platform of claim 1 is fabricated of Polydimethylsiloxane (PDMS), silicone or other flexible polymers and elastomers.

4. The biological membranes and tissue mentioned in claim 1, wherein human amniotic membrane, umbilical cord and placenta can be included in the platform.

5. The amniotic membrane mentioned in claim 4, wherein prior preparation of the membrane surface is done using chemical and mechanical methods to produce fully decellularized or partially decellularized surfaces based on application.

6. The three dimensional surface mentioned in claim 1, wherein patterns on the lining surface of the culture platform are made to mimic the microarchitecture and topography of the native in vivo cell niche.

7. The three dimensional surface mentioned in claim 1, wherein amniotic membrane based nano fibers are patterned in a network or aligned in various distribution to comprise the topography specific for the differentiating cells of interest.

8. The three dimensional surface mentioned in claim 1, wherein the surface topography can be patterned using three dimensional printers either for the prototype original material or for the amniotic membrane surface.

9. The three-dimensional surface mentioned in claim 1, wherein a free hanging piece can be inserted as a three dimensional disc for cell expansion on both sides.

10. The free hanging piece mentioned in claim 9, wherein the three dimensional surface is composed by the amniotic membrane and further enhanced by molding the amniotic membrane on configurations or arrays printed on the hanging piece.

11. As mentioned in claim #1, the cultured cells comprise proliferating, expanded or differentiated stem cells.

12. The stem cells mentioned in claim 11, comprising embryonic stem cells, adult stem cells, perinatal stem cells, fetal stem cells, cancer stem cells, induced pluripotent stem cells or any of their other subtypes.

13. The fluid dynamic factor mentioned in claim 1, comprising a method to create a continuously flowing or exchanged fluid surrounding the growing or differentiating cells.

14. The fluid mentioned in claim 13, wherein the fluid can be a culture medium, serum preferably human or cord blood derived, activated rich plasma or amniotic fluid.

15. The fluid dynamics mentioned in claim 1, wherein delivery of the fluid is through one or more inlet and outlet channels.

16. The inlet or outlet channels in claim 15, wherein channels can be stand free independent channels or can be microchannels of a microchip.

17. The outlet channels mentioned in claim 15, wherein such channels are equipped with one or more various size filters to prevent blocking of the channel by cells and to maintain a continuous flow.

18. The filters mentioned in claim 17, wherein filters can be external filters connected to the platform or internal built in posts inside micro channels.

19. The fluid dynamic factor mentioned in claim 1, wherein an external, portable or implantable pump system can be attached to the platform channels to automate the process of fluid exchange and suits in vivo stem cell applications.

20. The biomimetic platform mentioned in claim 1, wherein various cell culture platforms are used including microchips and lab-on-chip techniques.

21. The microchips mentioned in claim 20, wherein channel microfluidics platform or digital microfluidics platform is employed.

22. The microchips in platform mentioned in claim 20, wherein chips are made of modified and unmodified surfaces.

23. The biomimetic platform mentioned in claim 1, wherein the proposed platform can be used in stem cell research studies for a wide range of medical diseases.

24. The medical diseases mentioned in claim 23, wherein any medical dysfunction or disease model can be studied in particular cardiovascular diseases, neurological diseases, spinal cord injury, infections, cancer, diabetes, inflammatory, hormonal and immune disorders.

25. The biomimetic platform mentioned in claim 1, wherein an in vivo implantable model can be made by modifications of the research compatible version to suit therapeutic application.

26. The in vivo platform mentioned in claim 25, wherein stem cell based therapies can be delivered due to the low immunogenic properties of the amniotic membrane that support its use in transplantation therapies.

27. The in vivo platform mentioned in claim 25, wherein the human amniotic membrane is skeletonized in various shapes to create a cell carrier that can deliver undifferentiated or differentiated stem cells in vivo.
